(19) United States
(12) Patent Application Publication
(10) Pub. No.: US 2025/0161393 A1
(43) Pub. Date: May 22, 2025

(54) A PHARMACEUTICAL COMPOSITION FOR TREATING CANCER PAIN AND A PREPARATION METHOD THEREOF

(71) Applicant: Tianming Zhou, Yingtan, Jiangxi (CN)

(72) Inventor: Tianming Zhou, Yingtan, Jiangxi (CN)

(21) Appl. No.: 18/728,439

(22) PCT Filed: May 26, 2023
(86) PCT No.: PCT/CN2023/096579

§ 371 Date: Jul. 11, 2024

(30) Foreign Application Priority Data
Jul. 25, 2022 (CN) 202210884533.5

(51) Int. Cl. A61K 36/66; A61K 36/258; A61K 36/515; A61K 36/56; A61K 36/71; A61P 29/00

(52) U.S. Cl. CPC A61K 36/66; A61K 36/258; A61K 36/515; A61K 36/56; A61K 36/71; A61P 29/00

(57) ABSTRACT

A pharmaceutical composition for treating cancer pain and a preparation method thereof. The pharmaceutical composition is prepared from the following raw materials in parts by weight: 15-20 parts of raw Radix Aconiti Lateralis Praeparata, 6-10 parts of Herba Chelidonii, 5-8 parts of Radix et Rhizoma Notoginseng, 0.6-1 parts of Semen Strychni, 6-10 parts of raw Radix Aconiti Kusnezoffii, 1-2 parts of Gelsemium elegans. The preparation method of the pharmaceutical composition comprises the following steps: 1) firstly taking raw Radix Aconiti Lateralis Praeparata, Semen Strychni, raw Radix Aconiti Kusnezoffii in parts by weight, washing them, putting them in water, and decocting them for three hours to obtain the primary processing liquid. 2) Then taking Herba Chelidonii, Radix et Rhizoma Notoginseng, Gelsemium elegans in parts by weight, adding them to the obtained primary processing liquid, decocting twice, filtering after each decoction to get the filtrate; combining the filtrates obtained from the two times, thereby obtaining the decoction extract. 3) After the obtained decoction extract was left to settle for 24 hours, the pure liquid was obtained after filtration; then filtering it with a microporous membrane filter to obtain the filtrate; combining the pure liquid with the filtrate, and placing it into the concentrator for concentration treatment to obtain the final product.

### 1. TECHNICAL FIELD

The invention relates to a pharmaceutical composition, in particular to a pharmaceutical composition for treating cancer pain and a preparation method thereof.

### 2. BACKGROUND ART

The incidence of cancer is increasing year by year in China. Cancer pain, as one of the most common and painful symptoms in advanced malignant tumor patients. Cancer pain is caused by the cancer itself and the cancer treatment process (including surgery, radiotherapy, chemotherapy, etc.) and various factors such as mental, psychological, social, and economic factors. It often seriously affects the quality of life of patients. Patients may even commit suicide due to cancer pain. The World Health Organization and the International Association for the Study of Pain have developed the “three-step analgesic ladder” based on the current situation of cancer pain, which to some extent alleviates the pain of cancer patients.

However, because the clinical use of dolantin and morphine is restricted by various factors such as policies and regulations, society and humanities. Due to the drug resistance and side effects of dolantin and morphine, the control of cancer pain symptoms is still unsatisfactory. Cancer pain oral liquid has no side effects such as addiction and drug resistance of dolantin and morphine, and has good analgesic effect. The effect of retreatment for pain recurrence is still very good.

Therefore, although it may be an ideal pursuit goal to find a drug that can effectively relieve cancer pain without significant toxic and side effects, traditional Chinese medicine theory suggests that cancer pain is related to phlegm turbidity obstructing Qi and blood circulation. This provides a treatment approach for discovering traditional Chinese medicine to treat cancer pain. Therefore, the significances of the invention lie in fully utilizing the advantages of traditional Chinese medicine and combining with modern research results to develop traditional Chinese medicine as a collaborative method for treating cancer pain, and seeking more effective, safer drugs with less toxic and side effects.

### 3. SUMMARY OF THE INVENTION

The technical problem to be solved by the invention is to overcome the defect of the above technology and provide a pharmaceutical composition for treating cancer pain and a preparation method thereof.

In order to solve the above technical problems, the invention provides the following technical schem: a pharmaceutical composition for treating cancer pain. The pharmaceutical composition is prepared from the following raw materials in parts by weight: 15-20 parts of raw Radix Aconiti Lateralis Praeparata, 6-10 parts of Herba Chelidonii, 5-8 parts of Radix et Rhizoma Notoginseng, 0.6-1 parts of Semen Strychni, 6-10 parts of raw Radix Aconiti Kusnezoffii, 1-2 parts of Gelsemium elegans.

Fuerther, the pharmaceutical composition is prepared from the following raw materials in parts by weight: 15 parts of raw Radix Aconiti Lateralis Praeparata, 6 parts of Herba Chelidonii, 5 parts of Radix et Rhizoma Notoginseng, 0.6 parts of Semen Strychni, 6 parts of raw Radix Aconiti Kusnezoffii, 1 part of Gelsemium elegans.

Further, the pharmaceutical composition is prepared from the following raw materials in parts by weight: 18 parts of raw Radix Aconiti Lateralis Praeparata, 8 parts of Herba Chelidonii, 6 parts of Radix et Rhizoma Notoginseng, 0.8 parts of Semen Strychni, 8 parts of raw Radix Aconiti Kusnezoffii, 1 part of Gelsemium elegans.

Further, the pharmaceutical composition is prepared from the following raw materials in parts by weight: 20 parts of raw Radix Aconiti Lateralis Praeparata, 10 parts of Herba Chelidonii, 8 parts of Radix et Rhizoma Notoginseng, 1 part of Semen Strychni, 10 parts of raw Radix Aconiti Kusnezoffii, 2 parts of Gelsemium elegans.

The invention also provides a preparation method of the pharmaceutical composition for treating cancer pain, which comprises the following steps:
Step 1: Firstly taking raw Radix Aconiti Lateralis Praeparata, Semen Strychni, raw Radix Aconiti Kusnezoffii in parts by weight, washing them, putting them in water, and decocting them for three hours to obtain the primary processing liquid.
Step 2: Then taking Herba Chelidonii, Radix et Rhizoma Notoginseng, Gelsemium elegans in parts by weight, adding them to the primary processing liquid obtained in step 1, decocting twice, decocting one hour. During the first decoction, the amount of water added is 8-10 times of the total weight of the raw materials, decocting for 180 min, and the filtrate is obtained after filtering. During the second decoction, the amount of water added is 3-4 times of the total weight of raw materials, decocting for 60 min, and the filtrate is obtained after filtering. Combining the filtrates obtained from the two times, thereby obtaining the decoction extract.
Step 3: Adding 20% of 75% edible ethanol to the total amount of the decoction extract obtained in step 2, and letting it settle for 24 hours, then recovering the ethanol by distillation. The pure liquid was obtained after filtration; then filtering it with a microporous membrane filter to obtain the filtrate; combining the pure liquid with the filtrate, and placing it into the concentrator for concentration treatment to obtain the final product.

The sources of raw materials used in the invention are as follows:
Raw Radix Aconiti Lateralis Praeparata: pungent, sweet in taste, strongly hot in nature, toxic in property; the medicinal effect acts on heart, kidney and spleen; reviving Yang for resuscitation, supplementing “fire” to support Yang, expelling pathogenic wind, cold and dampness. Indications: Yang depletion and Qi deficiency, cold limbs with weak pulse, impotence, uterine coldness, cold pain in the epigastric region, deficiency-cold diarrhea and vomiting, edema due to Yin-cold, exogenous disease due to yang deficiency, rheumatic arthralgia. In addition, the raw Radix Aconiti Lateralis Praeparata contains aconitine, hypaconitine, mesaconitine and higenamine, which have analgesic and sedative effects.
Herba Chelidonii: bitter, pungent in taste, slightly warm in nature, toxic in property; the medicinal effect acts on lung, spleen, and stomach; analgesic, relieving cough and resolving phlegm, diuretic and detoxification. It is used for relieving pain, relieving cough, diuretic, detoxification, stomach pain, abdominal pain, enteritis, dysentery, chronic bronchitis, whooping cough, cough, jaundice, edema, ascites, swelling due to scabies, tinea and sores, snake or insect bites. In addition, Herba Chelidonii contains a variety of alkaloids, including Chelidonine, Protopine. Its roots and flowers also contain other alkaloids, which have antispasmodic and analgesic effects.
Radix et Rhizoma Notoginseng: sweet and slightly bitter in taste, warm in nature, slightly toxic in property; the medicinal effect acts on the liver, stomach, and large intestine. It is used to stop bleeding, disperse stasis, reduce swelling, and relieve pain. Indications: stopping bleeding, dispersing stasis, relieving pain, promoting blood circulation, reducing swelling. It can treat the pain and bleeding caused by gastric cancer, lung cancer, osteosarcoma, rectal cancer, and sigmoid colon cancer.
Semen Strychni: bitter in taste, cold in nature, slightly toxic in property; the medicinal effect acts on the liver and spleen; clearing heat from the blood, reducing swelling, relieving pain. It can treat esophageal cancer, gastric cancer, intestinal cancer, lung cancer, skin cancer, leukemia, etc. When applied externally, it can be used to treat skin cancer, subcutaneous nodule, throat obstruction, rheumatic arthralgia, bruises.
Raw Radix Aconiti Kusnezoffii: pungent, bitter in taste, hot in nature, toxic in property (Raw Radix Aconiti Kusnezoffii has strong toxin); the medicinal effect acts on the heart, liver, spleen and kidney; dispelling the wind and dampness, warming meridians to relieve pain. It is mainly used to treat arthralgia syndrome caused by wind-cold-dampness. In addition, the root tuber of raw Radix Aconiti Kusnezoffii contains aconitine, hypaconitine, and mesaconitine, which have analgesic effects.
Gelsemium elegans: pungent, bitter in taste, warm in nature, and extremely toxic in property.
Indications: dispelling wind and eliminating stasis, reducing swelling and relieving pain, attacking toxin and killing parasites. It can treat diseases such as rheumatic arthralgia, neuralgia, scrofula, bruises, fractures, hemorrhoids, etc.
Chemical composition: the root and whole herb contain indole alkaloids. The components that have been extracted currently include Gelsemine, N-methoxy-gelscmine, Sempervirine, Koumicine, Koumidine, Koumine, Kouminicine, Kouminidine, Kounidine, Gelsenicine, etc.
Pharmacological research: 1. Effects on the respiratory system: gelsemicine can inhibit respiration, but it is not central. Neither decerebrate nor cutting off the vagus nerve will affect its inhibition of respiration. Ephedrine and picrotoxin had no significant antagonistic effect on this.
2. Effects on peripheral nerves: gelsemicine can inhibit and stop the rhythmic contraction of the isolated diaphragm muscle with the phrenic nerve, which is similar to its effect on the in situ diaphragm muscle. Direct stimulation of the phrenic nerve can still cause the diaphragm to contract after breathing has ceased. It has no direct paralyzing effect on the peripheral nerve-muscle unit.
3. Effects on motor neurons: after the animals were poisoned, they all showed drooping eyelids, drooping heads, soft feet, and weak muscles in the whole body, so it is speculated that its effect is on the spinal cord. It can paralyze motor neurons.
4. Analgesic effect: gelsemine has an analgesic effect on mice, and its effective dose is close to the toxic dose. There are also reports that co-administration with aspirin can enhance the analgesic effect.
Commentary: Gelsemium elegans has the effects of dispelling wind and eliminating stasis, reducing swelling and relieving pain, attacking toxic and killing insects. It is an external medicine for treating bruises. It also has an analgesic effect on fractures, rheumatic arthralgia, and neuralgia. The toxicity of Gelsemium elegans is very strong, so oral administration should be strictly prohibited, and external use should also be carefully considered.
[Dosage of Gelsemium elegans]: It is obtained by the inventor himself after taking it for many times.

Compared with the prior art, the invention has the following advantages: the pharmaceutical composition for treating cancer pain provided by the invention has reasonable structure, precise compatibility, no obvious toxic and side effects, prevents cancer cell proliferation, metastasis and thrombus formation, and can inhibit the damage of chemical harmful factors such as inflammatory and painful factors, such as 5-hydroxytryptamine, released by cancer cells to normal cells and nerves. It also has the effect of promoting blood circulation and removing blood stasis, thereby achieving the therapeutic purpose of Chinese Medicine's Principle: bu tong ze tong, ze tong bu tong (it means that blockage of Qi and blood causes pain, while the flow of Qi and blood runs smoothly, it brings relief).

### 4. SPECIFIC EMBODIMENT OF THE INVENTION

A pharmaceutical composition for treating cancer pain and a preparation method thereof of the invention will be further described below in combination with embodiments.

### Embodiment 1

A pharmaceutical composition for treating cancer pain. The pharmaceutical composition is prepared from the following raw materials in parts by weight: 15 parts of raw Radix Aconiti Lateralis Praeparata, 6 parts of Herba Chelidonii, 5 parts of Radix et Rhizoma Notoginseng, 0.6 parts of Semen Strychni, 6 parts of raw Radix Aconiti Kusnezoffii, 1 part of Gelsemium elegans.

The preparation method of the pharmaceutical composition for treating cancer pain comprises the following steps:
Step 1: Firstly taking raw Radix Aconiti Lateralis Praeparata, Semen Strychni, raw Radix Aconiti Kusnezoffii in parts by weight, washing them, putting them in water, and decocting them for one hour to obtain the primary processing liquid.
Step 2: Then taking Herba Chelidonii, Radix et Rhizoma Notoginseng, Gelsemium elegans in parts by weight, adding them to the primary processing liquid obtained in step 1, decocting twice. During the first decoction, the amount of water added is 3-5 times of the total weight of the raw materials, decocting for 60 min, and the filtrate is obtained after filtering. During the second decoction, the amount of water added is 3-4 times of the total weight of raw materials, decocting for 50 min, and the filtrate is obtained after filtering. Combining the filtrates obtained from the two times, thereby obtaining the decoction extract.
Step 3: After the decoction extract obtained in step 2 was left to settle for 24 hours, the pure liquid was obtained after filtration; then filtering it with a microporous membrane filter to obtain the filtrate; combining the pure liquid with the filtrate, and placing it into the concentrator for concentration treatment to obtain the final product.

### Embodiment 2

A pharmaceutical composition for treating cancer pain. The pharmaceutical composition is prepared from the following raw materials in parts by weight: 18 parts of raw Radix Aconiti Lateralis Praeparata, 8 parts of Herba Chelidonii, 6 parts of Radix et Rhizoma Notoginseng, 0.8 parts of Semen Strychni, 8 parts of raw Radix Aconiti Kusnezoffii, 1 part of Gelsemium elegans.

The preparation method of the pharmaceutical composition for treating cancer pain comprises the following steps:
Step 1: Firstly taking raw Radix Aconiti Lateralis Praeparata, Semen Strychni, raw Radix Aconiti Kusnezoffii in parts by weight, washing them, putting them in water, and decocting them for one hour to obtain the primary processing liquid.
Step 2: Then taking Herba Chelidonii, Radix et Rhizoma Notoginseng, Gelsemium elegans in parts by weight, adding them to the primary processing liquid obtained in step 1, decocting twice. During the first decoction, the amount of water added is 3-5 times of the total weight of the raw materials, decocting for 60 min, and the filtrate is obtained after filtering. During the second decoction, the amount of water added is 3-4 times of the total weight of raw materials, decocting for 50 min, and the filtrate is obtained after filtering. Combining the filtrates obtained from the two times, thereby obtaining the decoction extract.
Step 3: After the decoction extract obtained in step 2 was left to settle for 24 hours, the pure liquid was obtained after filtration; then filtering it with a microporous membrane filter to obtain the filtrate; combining the pure liquid with the filtrate, and placing it into the concentrator for concentration treatment to obtain the final product.

### Embodiment 3

A pharmaceutical composition for treating cancer pain. The pharmaceutical composition is prepared from the following raw materials in parts by weight: 20 parts of raw Radix Aconiti Lateralis Praeparata, 10 parts of Herba Chelidonii, 8 parts of Radix et Rhizoma Notoginseng, 1 part of Semen Strychni, 10 parts of raw Radix Aconiti Kusnezoffii, 2 parts of Gelsemium elegans.

The preparation method of the pharmaceutical composition for treating cancer pain comprises the following steps:
Step 1: Firstly taking raw Radix Aconiti Lateralis Praeparata, Semen Strychni, raw Radix Aconiti Kusnezoffii in parts by weight, washing them, putting them in water, and decocting them for one hour to obtain the primary processing liquid.
Step 2: Then taking Gelsemium elegans, Herba Chelidonii, Radix et Rhizoma Notoginseng in parts by weight, adding them to the primary processing liquid obtained in step 1, decocting twice. During the first decoction, the amount of water added is 3-5 times of the total weight of the raw materials, decocting for 60 min, and the filtrate is obtained after filtering. During the second decoction, the amount of water added is 3-4 times of the total weight of raw materials, decocting for 50 min, and the filtrate is obtained after filtering. Combining the filtrates obtained from the two times, thereby obtaining the decoction extract.
Step 3: After the decoction extract obtained in step 2 was left to settle for 24 hours, the pure liquid was obtained after filtration; then filtering it with a microporous membrane filter to obtain the filtrate; combining the pure liquid with the filtrate, and placing it into the concentrator for concentration treatment to obtain the final product.

### Embodiment 4 Drug Toxicity Experiment

### 1. Experimental Materials

### 1.1 Experimental Animals:

44 ICR mice, half male and half male, were purchased from Xingwang Experimental Animal Farm in Haidian District of Beijing with license number SCXK-(Army) 2007-004.

### 1.2 Pharmaceutical Reagents

1) The pharmaceutical composition prepared by the invention is a liquid.

44 mice were randomly divided into experimental group and control group according to weight and gender, with 22 mice in each group, half male and half male. Before the experiment, they fasted but had free access to water for 12 hours.

The experimental group was administered the drug composition liquid prepared by the invention via gavage at a dose of 48 g/kg, and administered twice with an interval of 4 h.

The control group was given the same amount of normal water, and observed continuously for 7 days after administration. Observing and comparing the appearance, behavior, mental state, and respiratory changes of the two groups of mice every day, and weighing once every other day.

The results showed that within 7 days after administration, the animals did not die, and were in good general condition, with normal coat color, no piloerection, normal diet, normal urination, dry stool, no diarrhea or loose stools, normal breathing, free movement, and no abnormal behavior.

The weight of the mice grew normally, and no other obvious and abnormal reactions were seen. They were sacrificed and dissected. No obvious abnormalities were found in the main organs, so the drug belongs to the non-toxic grade.

### Embodiment 5 Animal Pain Relief Experiment

### 1. Experimental Materials

### 1.1 Experimental Animals:

90 ICR mice, both male and female, were purchased from

Xingwang Experimental Animal Farm in Haidian District of Beijing with license number SCXK-(Army) 2007-004.

### 1.2 Pharmaceutical Reagents

1) The pharmaceutical composition prepared by the invention;
2) Positive control drug: commercially available analgesic drugs;
3) Glacial acetic acid (HAc), analytically pure.

### 2. Experimental Method

### 2.1 Reagent Preparation

Prepare 0.6% glacial acetic acid with normal saline solution. The specific method: accurately absorbing 0.3ml of 100% glacial acetic acid and dissolving it in 50ml of normal saline solution with a pH of 7-7.2.

2.2

90 experimental mice were equally divided into control group, commercially available analgesic drug group (67 mg/kg), large, medium and small dose groups of pharmaceutical composition (respectively 8.4 g/kg, 4.2 g/kg, 2.1 g/kg, which were 20 times, 10 times, and 5 times the dosage of clinical human), 18 animals in each group were given the equal volume of solvent, commercially available analgesic drugs and different doses of pharmaceutical composition oral liquid.

One hour after administration to each experimental group, 0.6% HAc normal saline solution at 0.2 mL per mouse was injected into the abdominal cavity. Observing the mice for 30 minutes for writhing response times (abdominal constrictions, extensions of hind legs, and buttocks raising) and writhing response rates, recording the results. Calculate the analgesic percentage of each group according to the formula, wherein the administration group includes commercially available analgesic drugs group, pharmaceutical composition large, medium and small dose groups.

$$Percentage of analgesic response = \frac{\begin{matrix}Number of writhing responses in the control group - \\ Number of writhing reactions in the administration group\end{matrix}}{Number of writhing responses in the control group} \times 100 \%$$

Statistical analysis was performed by using SPSS software for t-test between groups. The results showed that commercially available analgesic drugs, large, medium and small doses of the pharmaceutical composition could significantly reduce the number of writhing responses in mice induced by glacial acetic acid (p<0.05, p<0.01), and analgesic effect increased with the increase of drug dosage.

### Embodiment 6 Clinical Experiment

### 1.1 Experimental Content

In this group of 40 patients with cancer pain, there were 28 males and 12 females, the youngest age was 28 years old, the oldest age was 72 years old, and the average age was 50 years old. Among them, there were 10 cases of lung cancer, 16 cases of liver cancer, 8 cases of gastric cancer, 6 cases of pancreatic cancer, 22 cases of moderate pain, and 18 cases of severe pain. All cases were diagnosed by MRI, gastroscope, CT and pathological examination, and the diagnoses were confirmed. Patients had pain from moderate to severe. They had no obvious liver or kidney dysfunction. That have clear consciousness and can cooperate with observation and treatment.

Medication method: three times a day, 20 ml each time, orally on a half-empty stomach (two hours after meals or between meals).

### 1.2 Pain Classification Criteria

According to the literature, the pain is divided into 4 grades:
Grade 0: no pain;
Grade I: (mild pain) the pain is tolerable, but does not affect daily production and sleep;
Grade II: (moderate pain), the pain is obvious and unbearable, daily life and sleep are affected, and analgesics are required;
Grade III: (severe pain), the pain is intense, intolerable, analgesics are required, and sleep is severely disturbed.

### 1.3 Response Evaluation Criteria

The degree of response after treatment is generally divided into 4 grades:
1) Complete response (CR): no pain;
2) Partial response (PR): the pain is significantly reduced compared with that before the administration, the sleep is basically undisturbed, and the patient can live normally;
3) Marginal response (MR): the pain is reduced compared with that before the administration, but there is obvious pain, and sleep is disturbed;
4) No response (NR): no pain relief compared with that before the administration.

### 1.4 Experimental Results

After taking Aitongding Oral Liquid, 22 cases began to relieve pain within 8-12 hours until the pain disappeared, the CR rate was 55%; 9 cases had partial response, the PR rate was 22.5%; 5 cases had marginal response, the MR rate was 12.5%; 4 cases had no response, the NR rate was 10%. The overall response rate was 90%.

The fastest time to achieve effect is 8 hours, the slowest is 24 hours, and the average is 16 hours. The onset age, pain type and degree were comparable between the two groups (P>0.05).

The invention and its embodiments are described above. This description is not restrictive. All in all, if those skilled in the art receives its enlightenment, without deviating from the object of the invention, and without creatively designing structures and embodiments similar to the technical scheme of the invention shall fall within the protection scope of the invention.

1. A pharmaceutical composition for treating cancer pain; the pharmaceutical composition is prepared from the following raw materials in parts by weight: 15-20 parts of raw Radix Aconiti Lateralis Praeparata, 6-10 parts of Herba Chelidonii, 5-8 parts of Radix et Rhizoma Notoginseng, 0.6-1 parts of Semen Strychni, 6-10 parts of raw Radix Aconiti Kusnezoffii, 1-2 parts of Gelsemium elegans.

2. The pharmaceutical composition for treating cancer pain of claim 1, the pharmaceutical composition is prepared from the following raw materials in parts by weight: 15 parts of raw Radix Aconiti Lateralis Praeparata, 6 parts of Herba Chelidonii, 5 parts of Radix et Rhizoma Notoginseng, 0.6 parts of Semen Strychni, 6 parts of raw Radix Aconiti Kusnezoffii, 1 part of Gelsemium elegans.

3. The pharmaceutical composition for treating cancer pain of claim 1, the pharmaceutical composition is prepared from the following raw materials in parts by weight: 18 parts of raw Radix Aconiti Lateralis Praeparata, 8 parts of Herba Chelidonii, 6 parts of Radix et Rhizoma Notoginseng, 0.8 parts of Semen Strychni, 8 parts of raw Radix Aconiti Kusnezoffii, 1 part of Gelsemium elegans.

4. The pharmaceutical composition for treating cancer pain of claim 1, the pharmaceutical composition is prepared from the following raw materials in parts by weight: 20 parts of raw Radix Aconiti Lateralis Praeparata, 10 parts of Herba Chelidonii, 8 parts of Radix et Rhizoma Notoginseng, 1 part of Semen Strychni, 10 parts of raw Radix Aconiti Kusnezoffii, 2 parts of Gelsemium elegans.

5. The preparation method of the pharmaceutical composition for treating cancer pain of claim 1, which comprises the following steps:
Step 1: Firstly taking raw Radix Aconiti Lateralis Praeparata, Semen Strychni, raw Radix Aconiti Kusnezoffii in parts by weight, washing them, putting them in water, and decocting them for three hours to obtain the primary processing liquid;
Step 2: Then taking Herba Chelidonii, Radix et Rhizoma Notoginseng, Gelsemium elegans in parts by weight, adding them to the primary processing liquid obtained in step 1, decocting twice, decocting one hour. During the first decoction, the amount of water added is 8-10 times of the total weight of the raw materials, decocting for 180 min, and the filtrate is obtained after filtering; during the second decoction, the amount of water added is 3-4 times of the total weight of raw materials, decocting for 60 min, and the filtrate is obtained after filtering. Combining the filtrates obtained from the two times, thereby obtaining the decoction extract;
Step 3: Adding 20% of 75% edible ethanol to the total amount of the decoction extract obtained in step 2, and letting it settle for 24 hours, then recovering the ethanol by distillation;
the pure liquid was obtained after filtration; then filtering it with a microporous membrane filter to obtain the filtrate; combining the pure liquid with the filtrate, and placing it into the concentrator for concentration treatment to obtain the final product.
